(19) United States
(12) Patent Application Publication
(10) Pub. No.: US 2012/0238954 A1
(43) Pub. Date: Sep. 20, 2012

(54) EXPANDABLE APPARATUS FOR MEDICAL TREATMENT

(75) Inventors: Chang-Hwa YOU, Gunpo-si (KR); Boo-Gou PARK, Incheon (KR)

(73) Assignee: BMKOREA CO., LTD, Hwaseong-si (KR)

(21) Appl. No.: 13/096,247

(22) Filed: Apr. 28, 2011

(30) Foreign Application Priority Data
Mar. 14, 2011 (KR) 10-2011-0022211

(51) Int. Cl. A61M 25/10

(52) U.S. Cl. 604/103.1

(57) ABSTRACT

An expandable apparatus for medical treatment is provided. The expandable apparatus includes an expandable member, an air tube, a connector, a main body, a front cap and a long needle. The air tube guides air into the expandable member. The connector is coupled to the air tube to guide air into the air tube. The main body is coupled to the connector and includes an air inlet pipe into which the air for expanding the expandable member is supplied. The front cap is coupled to the main body such that the main body supports the connector. The front cap protects a junction between the air tube and the connector. The long needle extends from a rear end of the main body to the expandable member. The long needle supports the expandable member and the air tube. A marker is integrally provided on the long needle corresponding to a position of the expandable member.

### PRIORITY

This application claims the benefit under 35 U.S.C. §119(a) of a Korean patent application filed on Mar. 14, 2011 in the Korean Intellectual Property Office and assigned Serial No. 10-2011-0022211, the entire disclosure of which is hereby incorporated by reference.

### BACKGROUND OF THE INVENTION

1. Field of the Invention

The present invention relates to expandable apparatuses for medical treatment. More particularly, the present invention relates to an expandable apparatus for medical treatment which includes an expandable member having a high modulus of elasticity and superior expansibility which is used to detach adhered portions of nervous tissue or muscular tissue from each other or to form space through which liquid treatment material is injected onto the body tissue of a lesion, and which includes a long needle having an increased thickness to more reliably support the air tube and the expandable member so that the air tube is prevented from moving, and in which a marker is integrally formed on the long needle, thus preventing a problem of a conventional art in which a separate marker provided on the long needle may be undesirably removed therefrom and thus put the patient in danger.

2. Description of the Related Art

Generally, human bodies have fine blood vessels and small organs which may be easily damaged even by light impact. Further, events frequently occur in which such a fine blood vessel or a small organ is clogged by a foreign substance. Particularly, most of these events are emergencies and the foreign substance must be removed as soon as possible. Furthermore, if an adhesion of a fine blood vessel or a small organ occurs because of inflammation or cancer, the function thereof deteriorates, and it is difficult to correctly apply the medicine that treats the lesion. These problems cause the administration of an excessive amount of antibiotics or anticancer medicine.

Meanwhile, in the dental field, in the case of periodontal bone fusion, nerves or a nerve and a tissue occasionally adhere to each other due to necrosis of a periodontal bone in a gum located on an upper jaw joint. In the conventional art, metal apparatuses or equipment using high heat have been used to treat these adhesions. However, accidents frequently occurred in which the conventional apparatuses or equipment damaged the adhered nerves in the upper jaw joint, thus causing facial paralysis of a patient or death.

With regard to the emergency medical field, there is occasionally an accident wherein the airway is obstructed attributable to a foreign substance. In this case, the patient has trouble breathing and, in more serious cases the patient may die. Therefore, rapid emergency treatment is required. In the conventional art, bronchial dilatation has been implemented with metal apparatuses. However, the esophagus of the patient may be damaged by a metal apparatus. Depending on the kind of foreign substance or the depth of the foreign substance in the esophagus, the metal apparatus may not be able to be used.

Furthermore, in the internal medicine department dealing with kidneys, urethral canal dilatation is required when a calculus moves down into the bladder and sticks in the urethral canal during urination. In this case, the pain is too severe for a patient to walk. In the conventional art, the urinary tract is cut to remove the calculus from the urethral canal, or extracorporeal equipment using shock waves is used. However, a scar may be left or the urinary tract may be damaged. In addition, the relapse rate is comparatively high.

Further, in the spinal surgery department, when a vertebral body compression fracture occurs, for example, due to osteoporosis, an expandable apparatus has been used to secure a space for putting bone cement into the fractured spinal segment. However, due to the movement of an air tube of the expandable apparatus, it is not easy to insert an expandable member into a desired portion. Sometimes a marker may be undesirably removed form the expandable apparatus, and the removed marker may cause an inflammation in the patient's body.

To overcome the above problems, a variety of apparatuses having expandable structures have been proposed. Such an apparatus having an expandable structure is connected to a manual or automatic pressure generator so that the size or pressure of an expandable member can be controlled by air pressure generated by the pressure generator in response to the size of a blood vessel or the force with which nervous tissue or muscular tissue is adhered to each other. The expandable apparatus using this principle is used to detach the adhered portions of the nervous tissue or muscular tissue from each other or to secure a space so that a desired portion can be filled with liquid treatment material. Moreover, when an organ has been clogged by a foreign substance, the expandable apparatus is used to expand the clogged organ so that the foreign substance easily moves away from the organ. As necessary, a mixture of distilled water and contrast media may be used in the pressure generator to determine the location and the size of the expandable member. Furthermore, after the problems mentioned above have been solved, a liquid treatment material, such as antiseptic or bone cement, may be used to treat damaged tissue, when necessary.

FIG. 1 is a schematic sectional view showing an expandable apparatus for medical treatment according to the related art.

Referring to FIG. 1, the expandable apparatus includes a main body 15 to which an air tube 12 is coupled. An auxiliary tube 13 is disposed in the air tube 12. A long needle 18 is provided in the auxiliary tube 13. Furthermore, a rear cap 17 is coupled to the main body 15, and the long needle 18 is coupled to the rear cap 17. The main body 15 has an air inlet pipe 16. The air inlet pipe 16 is connected to an external air supply device (not shown) by an air supply tube 21.

However, the conventional expandable apparatus for medical treatment has the following problems.

In the conventional expandable apparatus, the air tube 12 is coupled to the main body 15, but the air tube 12 is not reliably coupled to the main body 15 at a coupling junction 10 therebetween. Thus, the air tube 12 may undesirably detach itself from the main body 15, so that expansion of an expandable member (not shown) cannot be implemented. Furthermore, the long needle 18 made of metal is fastened to the rear cap 17, and the rear cap 17 is separably coupled to the main body 15, for example, by threaded coupling. In this structure, if the rear cap 17 is not firmly coupled to the main body, the long needle 18 made of metal does not align correctly with the air tube 12, thus damaging the air tube 12, for example, tearing the air tube 12.

In addition, a separate marker, such as a platinum ring or the like, for indicating the location of the expandable member is coupled to a distal end of the long needle 18 by a method, such as welding. However, there is a problem in that the marker may be removed from the long needle by factors, such as defective welding, external shock, or other surrounding factors. As such, if the marker is removed from the long needle, the location of the expandable member cannot be exactly indicated. Moreover, if the air tube or the expandable member tears, the removed marker may come out of the air tube or the expandable member and enter the patient's body, thus causing the danger of, for example, the occurrence of an inflammation.

### SUMMARY OF THE INVENTION

Aspects of the present invention are to address at least the above-mentioned problems and/or disadvantages and to provide at least the advantages described below. Accordingly an aspect of the present invention is to provide an expandable apparatus for medical treatment which includes an expandable member having a high modulus of elasticity and superior expansibility which is used to detach adhered portions of nervous tissue or muscular tissue from each other or to form space through which liquid treatment material is injected onto the bodily tissue of a lesion, and which includes a long needle having an increased thickness to more reliably support the air tube and the expandable member so that the air tube is prevented from moving, and in which a marker is integrally formed on the long needle, thus preventing a problem of a conventional art in which a separate marker provided on the long needle may be undesirably removed therefrom and thus put a patient in danger.

In accordance with an aspect of the present invention, an expandable apparatus for medical treatment is provided. The apparatus includes an expandable member inserted into a body of a patient and expanded by air pressure, an air tube guiding air into the expandable member to expand the expandable member, the air tube having a first end coupled to the expandable member to support the expandable member, a connector guiding the air into the air tube, the connector being closely coupled to the air tube in such a way that a first end of the connector is inserted into a second end of the air tube, a main body comprising an air inlet pipe into which the air for expanding the expandable member is supplied from an outside, the main body having a first end coupled to a second end of the connector so that the air supplied into the air inlet pipe is transferred to the connector, a front cap coupled to the first end of the main body such that the main body supports the connector while the connector is brought into close contact with the main body by the front cap, the front cap protecting a coupling junction between the air tube and the connector, and a long needle extending from a second end of the main body to the expandable member through the main body, the connector and the air tube, the long needle assisting in supporting the expandable member and the air tube, with a marker integrally provided on a first end of the long needle corresponding to a position of the expandable member, the marker enabling a user to determine a location of the expandable member in the body of the patient.

In an exemplary implementation, the connector may be welded to the air tube.

Furthermore, the connector may have an inner passage through which the air passes. The inner passage may have a tapered structure so that the expandable member expands at a pressure equal to a pressure of the air when supplied into the main body.

The expandable apparatus may further include an O-ring interposed between the main body and the connector to prevent the air from leaking.

In addition, the expandable apparatus may further include a rear cap coupled to the second end of the main body, the rear cap supporting the long needle. The long needle may be fastened to the rear cap and the rear cap may be welded to the main body so that the long needle is prevented from damaging the air tube.

In accordance with an aspect of the present invention, an expandable apparatus for medical treatment is provided. The expandable apparatus includes a connector which is closely coupled to an air tube and has a tapered internal structure, and a front cap which closely couples the connector to a main body. Thus, the air tube can smoothly guide air from the main body into an expandable member to expand the expandable member. Therefore, the expandable apparatus can reliably prevent the expandable member from losing its expanding function.

Furthermore, a rear cap to which the long needle is fastened is welded to the main body. Thus, the air tube can be prevented from being damaged by the long needle, thereby enhancing the stability of the expandable apparatus.

In addition, a syringe or the like can be directly connected to an air inlet pipe of the main body so that in an emergency the expandable member can be more rapidly expanded.

Moreover, a marker is integrally formed on a distal end of the long needle, thus fundamentally preventing the marker from being removed from the long needle. Further, because the long needle has a thickness similar to that of the air tube, movement of the air tube can be reduced while the location of the expandable member is determined.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features, and advantages of certain exemplary embodiments of the present invention will be more apparent from the following description taken in conjunction with the accompanying drawings, in which:

FIG. 1 is a schematic sectional view showing an expandable apparatus for medical treatment according to the related art;

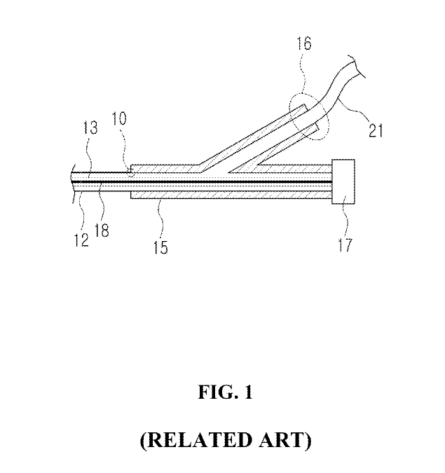

FIG. 2 is a schematic sectional view showing an expandable apparatus for medical treatment, according to an exemplary embodiment of the present invention;

FIG. 3 is a partial sectional view of the expandable apparatus according to an exemplary embodiment of the present invention;

FIG. 4 is a sectional view showing a connector of the expandable apparatus according to an exemplary embodiment of the present invention; and

FIG. 5 is a sectional view showing an expanded state of an expandable member of the expandable apparatus according to an exemplary embodiment of the present invention.

Throughout the drawings, it should be noted that like reference numbers are used to depict the same or similar elements, features, and structures.

### DETAILED DESCRIPTION OF EXEMPLARY EMBODIMENTS

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

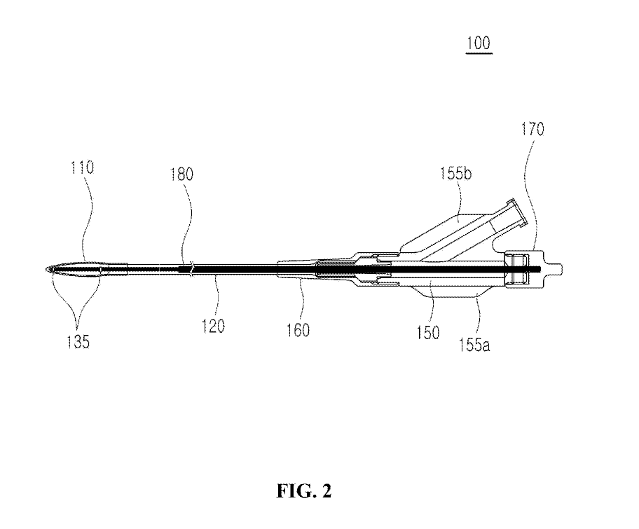

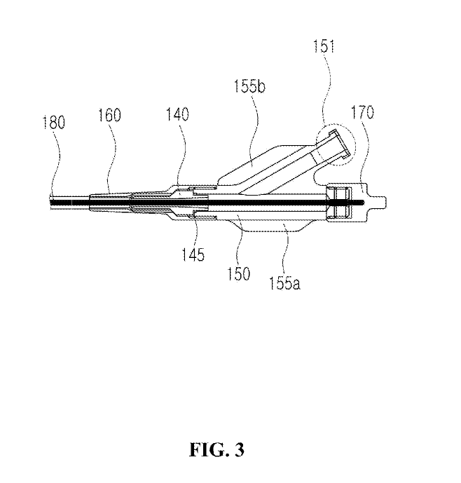

FIG. 2 is a schematic sectional view showing an expandable apparatus 100 for medical treatment, according to an exemplary embodiment of the present invention. FIG. 3 is a partial sectional view of the expandable apparatus 100 according to an exemplary embodiment of the present invention. As shown in FIGS. 2 and 3, the expandable apparatus 100 includes an expandable member 110, an air tube 120, a connector 140, a main body 150, a front cap 160, a long needle 180 and a rear cap 170.

The main body 150 transmits air from an external pneumatic pressure generator, such as a syringe, into the connector 140. The main body 150 includes an air inlet pipe 151 into which air for expanding the expandable member 110 is supplied. A first end of the main body 150 is coupled to a second end of the connector 140.

Air is supplied from the external pneumatic pressure generator, such as an air syringe, into the main body 150 through the air inlet pipe 151. The air that is supplied into the main body 150 enters the expandable member 110 via the connector 140 and the air tube 120 to expand the expandable member 110.

Preferably, a thread (not shown) is formed on the air inlet pipe 151 so that the air syringe (not shown) is coupled to the air inlet pipe 151 by thread-coupling. When the syringe is directly coupled to the air inlet pipe 151, the expandable member 110 can be more rapidly expanded, and a separate air pipe between the main body 150 and the syringe is not required. Furthermore, because the syringe is directly connected to the main body 150 without using the air pipe, a reduction in air pressure due to a tapered internal structure of the connector 140 can be minimized, so that sufficient air pressure can be applied to the expandable member 110.

In addition, it is preferable that handles 155a and 155b be provided on the main body 150 to enable a user to easily handle the expandable apparatus.

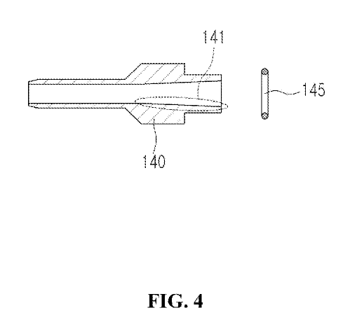

FIG. 4 is a sectional view showing the connector 140 of the expandable apparatus 100 according to an exemplary embodiment of the present invention.

Referring to FIG. 4, the connector 140 guides air supplied into the main body 150 from the outside so that the air enters the air tube 120. Furthermore, the connector 140 is closely coupled to the air tube 120 and thus supports the air tube 120. In detail, a first end of the connector 140 is inserted into a second end of the air tube 120 such that the connector 140 is closely coupled to the air tube 120. As shown in FIG. 4, the connector 140 is preferably tapered from the second end thereof to the first end. In other words, the connector 140 is tapered to the first end thereof such that the first end of the connector 140 is easily inserted into the second end of the air tube 120. As such, because the first end of the connector 140 is disposed in the second end of the air tube 120, the connector 140 can more reliably support the air tube 120. Furthermore, after the first end of the connector 140 is inserted into the second end of the air tube 120, if the junction between the connector 140 and the air tube 120 is sealed, for example, by welding, air leakage can be more effectively prevented.

That is, to prevent air from leaking, it is preferable that the coupling between the air tube 120 and the connector 140 take the form of welding. More preferably, all of the connector 140, the air tube 120 and the expandable member 110 are welded to each other.

The connector 140 has an inner passage through which air passes. The inner passage is tapered so that the expandable member 110 can expand at a pressure equal to the pressure of air when it is supplied into the main body 150. As such, because the inner passage of the connector 140 is tapered, the pressure of air that is reduced after the air has been supplied into the main body 150 can be increased again when the air is drawn into the expandable member 110 via the air tube 120.

In an exemplary implementation, an O-ring 145 is interposed between the connector 140 and the main body 150 to prevent fluid, that is, air, from leaking therefrom. Furthermore, the front cap 160 is coupled to the main body 150 so that the connector 140 is closely coupled to the main body 150, thus more reliably preventing fluid, such as air, from leaking therefrom.

Meanwhile, when the expandable member 110 expands, distilled water or contrast media (not shown) must be rapidly supplied to the expandable member 110 to enable the user to discern the size to which it expands or the expanded state thereof. In an exemplary embodiment of the present invention, the connector 140 is integrally coupled to the air tube 120 and has the internal tapered structure 141, so that the pressure of contrast media that is reduced when supplied into the main body 150 can be increased by the internal tapered structure 141. Thus, the contrast media can be rapidly transferred towards the expandable member 110. Moreover, the internal tapered structure of the connector 140 also functions to minimize the pressure of the fluid flowing backwards from the expandable member 110.

In an exemplary embodiment of the present invention, the front cap 160 is coupled to the first end of the main body 150 such that the connector 140 is closely coupled to the main body 150. Further, the front cap 160 supports the connector 140. Preferably, the front cap 160 is tapered at the junction between the front cap 160 and the connector 140 so that the coupling of the connector 140 to the main body 150 is effective and close.

In addition, the front cap 160 also functions to protect the coupling portion, that is, the junction, between the air tube 120 and the connector 140.

The air tube 120 guides air from the connector 140 into the expandable member 110 so that the expandable member 110 can be expanded by the air pressure. Furthermore, the air tube 120 supports the expandable member 110. Preferably, a first end of the air tube 120 is fused to the expandable member 110 to be coupled thereto and to prevent air from leaking therebetween.

The long needle 180 is inserted into the air tube 120. A second end of the air tube 120 is coupled to the connector 140. Preferably, the second end of the air tube 120 is coupled to the connector 140 by fusing to prevent air from leaking between the connector 140 and the air tube 120. The air tube 120 is supported by the connector 140.

The expandable member 110 is expanded in the body of a patient by the pressure of air supplied thereinto from the air tube 120. In detail, the expandable member 110 is inserted into the patient's body, for example, nervous tissue, muscular tissue, etc., and then expended so that adhered portions of the nervous tissue are detached from each other, the nervous tissue and the muscular tissue which were adhered to each other are detached from each other, or a space is formed for injecting treatment material into the tissues of the patient's body. The expandable member 110 is coupled to the first end of the air tube 120. Air is supplied into the expandable member 110 from the air tube 120 to expand the expandable member 110.

It is preferable that such an expandable member 110 be made of TPU (thermoplastic-polyurethane) that has a high modulus of elasticity and superior expansibility.

The long needle 180 functions to support the expandable member 110 and the air tube 120. Furthermore, the long needle 180 extends to the expandable member 110 through the main body 150, the connector 140 and the air tube 120. A second end of the long needle 180 is fastened to the rear cap 170. The long needle 180 supports and guides the expandable member 110 to allow the user to insert the expandable member 110 into a desired location in the patient's body. Air is supplied from the outside into the expandable member 110 through a space defined between the long needle 180 and the air tube 120, thus expanding the expandable member 110. In the embodiment, markers 135 integrally protrude on a first end of the long needle 180, preferably, on a predetermined portion of the first end of the long needle 180 that corresponds to the position of the expandable member 110, so that the user can easily determine the location of the expandable member 110 using the markers 135. The markers 135 which are provided on the predetermined portion of the first end of the long needle 180 are disposed inside the expandable member 110.

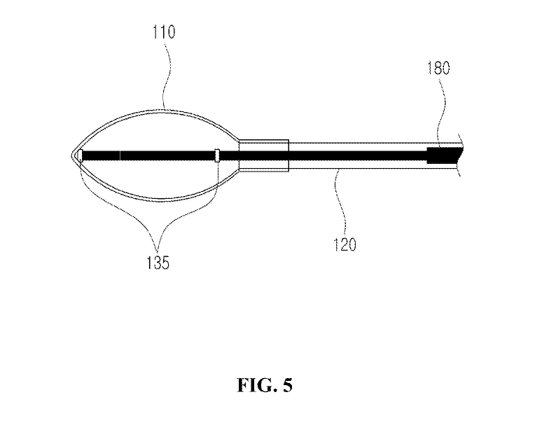

FIG. 5 is a sectional view showing an expanded state of the expandable member 110 of the expandable apparatus 100 according to an exemplary embodiment of the present invention. In an embodiment, when the user operates a radiation apparatus, such as a C-RAM, to apply radiation to a related portion of the patient's body, as shown in FIG. 5, the markers 135 provided on the first end of the long needle 180 reflect the radiation so that the location of the expandable member 110 can be determined by the radiation that is reflected by the markers 135. In the conventional art, such a marker comprises a platinum ring or the like which is manufactured in a separate process and coupled to the long needle 180 by a method, such as welding. However, in an exemplary embodiment of the present invention, because the markers 135 are formed by processing the first end of the long needle 180, the markers 135 can be prevented from being removed from the long needle 180.

The long needle 180 having such structure has an outer diameter less than an inner diameter of the air tube 120 so that fluid can flow through the air tube 120. The first end of the long needle 180 is processed, for example, by cutting such that the markers 135 protrude from the first end of the long needle 180.

Meanwhile, the rear cap 170 supports the long needle 180. In detail, the rear cap 170 is coupled to the second end of the main body 150. Preferably, the rear cap 170 is welded to the second end of the main body 150. Furthermore, the long needle 180 which is fastened to the rear cap 170 extends to the expandable member 110 through the air tube 120 and supports the air tube 120. The long needle 180 assists in supporting the air tube 120 and the expandable member 110. It is preferable that the long needle 180 be made of elastic metal.

Because the rear cap 170 to which the long needle 180 is fastened is welded to the main body 150, aligning the long needle 180 with the air tube 120 can be facilitated, thus preventing the air tube 120 from being damaged by the long needle 180.

As described above, the expandable apparatus 100 includes the connector 140 which is closely coupled to the air tube 120 and has the tapered internal structure, and the front cap 160 which closely couples the connector 140 to the main body 150. Therefore, the air tube 120 can smoothly supply air from the main body 150 into the expandable member 110 to expand the expandable member 110. Thus, the exemplary embodiment can prevent the expandable member 110 from losing its expansionary function.

Furthermore, in the expandable apparatus 100, the rear cap 170 to which the long needle 180 is fastened is welded to the main body 150. Thus, the air tube 120 can be prevented from being damaged by the long needle 180, thereby enhancing the stability of the expandable apparatus. In addition, a syringe or the like can be directly connected to the air inlet pipe 151 of the main body 150 so that in an emergency the expandable member 110 can be more rapidly expanded.

While the invention has been shown and described with reference to certain exemplary embodiments thereof, it will be understood by those skill in the art that various changes in form and details may be made therein without departing from the spirit and scope of the invention as defined by the appended claims and their equivalents.

What is claimed is:

1. An expandable apparatus for medical treatment, the apparatus comprising:
an expandable member inserted into a body of a patient and expanded by air pressure;
an air tube guiding air into the expandable member to expand the expandable member, the air tube having a first end coupled to the expandable member to support the expandable member;
a connector guiding the air into the air tube, the connector being closely coupled to the air tube in such a way that a first end of the connector is inserted into a second end of the air tube;
a main body comprising an air inlet pipe into which the air for expanding the expandable member is supplied from an outside, the main body having a first end coupled to a second end of the connector so that the air supplied into the air inlet pipe is transferred to the connector;
a front cap coupled to the first end of the main body such that the main body supports the connector while the connector is brought into close contact with the main body by the front cap, the front cap protecting a coupling junction between the air tube and the connector; and
a long needle extending from a second end of the main body to the expandable member through the main body, the connector and the air tube, the long needle assisting in supporting the expandable member and the air tube, with a marker integrally provided on a first end of the long needle corresponding to a position of the expandable member, the marker enabling a user to determine a location of the expandable member in the body of the patient.

2. The expandable apparatus as set forth in claim 1, wherein the connector is welded to the air tube.

3. The expandable apparatus as set forth in claim 1, wherein the connector has an inner passage through which the air passes, the inner passage having a tapered structure so that the expandable member expands at a pressure equal to a pressure of the air when supplied into the main body.

4. The expandable apparatus as set forth in claim 1, further comprising:
an O-ring interposed between the main body and the connector to prevent the air from leaking.

5. The expandable apparatus as set forth in claim 1, further comprising:
a rear cap coupled to the second end of the main body, the rear cap supporting the long needle,
wherein the long needle is fastened to the rear cap and the rear cap is welded to the main body so that the long needle is prevented from damaging the air tube.
